(19) United States
(12) Patent Application Publication
(10) Pub. No.: US 2022/0152599 A1
(43) Pub. Date: May 19, 2022

(54) CATALYST FOR CONVERTING ETHANE TO MONOAROMATIC HYDROCARBONS

(71) Applicant: SABIC Global Technologies B.V., Bergen Op Zoom (NL)

(72) Inventors: Suman Kumar Jana, Bangalore (IN); Selvakumar Subramanian, Bangalore (IN); Deepa Govindarajan, Bangalore (IN); Ziyad Kottavarithottil, Bangalore (IN); Amit Kumar, Bangalore (IN); Ninad Loke, Bangalore (IN); SreenivasaRao Gajula, Bangalore (IN)

(21) Appl. No.: 17/389,732

(22) Filed: Jul. 30, 2021

Related U.S. Application Data
(60) Provisional application No. 63/114,058, filed on Nov. 16, 2020.

(51) Int. Cl. B01J 29/44; B01J 37/08; B01J 37/02; B01J 23/63; B01J 37/00; B01J 37/04; B01J 35/10; B01J 35/02; C07C 2/84

(52) U.S. Cl. CPC B01J 29/44; B01J 37/082; B01J 37/024; B01J 23/63; B01J 37/0009; C07C 2529/40; B01J 35/1019; B01J 35/023; C07C 2/84; B01J 2229/18; C07C 2529/44; B01J 37/04

(57) ABSTRACT

A catalyst for converting ethane to monoaromatic hydrocarbons including: a zeolite; cesium oxide, wherein cesium of the cesium oxide is present in an amount of 0.01 to 0.5 weight percent, preferably 0.01 to 0.1 weight percent, more preferably 0.03 to 0.07 weight percent, based on a total weight of the catalyst; platinum oxide, wherein platinum of the platinum oxide is present in an amount of 0.01 to 1 weight percent, preferably 0.01 to 0.5 weight percent, more preferably 0.01 to 0.05 weight percent, based on a total weight of the catalyst; and gallium oxide, wherein gallium of the gallium oxide is present in an amount of 0.01 to 1 weight percent, preferably 0.03 to 0.5 weight percent, more preferably 0.05 to 0.2 weight percent, based on a total weight of the catalyst; wherein the monoaromatic hydrocarbons include benzene, toluene, xylene, or a combination including at least one of the foregoing.

### CROSS-REFERENCE TO RELATED APPLICATIONS

This application claims priority to and the benefit of U.S. Provisional Application No. 63/114,058 filed on Nov. 16, 2020, the entire content of which is incorporated herein by reference.

### BACKGROUND

Ethane can be converted to monoaromatic hydrocarbons (e.g., benzene, toluene and xylenes—“BTX” aromatics) via the aromatization of ethane. In general, the conversion of ethane to monoaromatic hydrocarbons can be a multi-step chemical transformation, and can require a catalyst.

An interest exists for improved catalysts for converting ethane to monoaromatic hydrocarbons.

These and other inefficiencies and opportunities for improvement are addressed and/or overcome by the processes, methods and catalysts of the present disclosure.

### BRIEF DESCRIPTION

The present disclosure provides advantageous catalysts for converting ethane to monoaromatic hydrocarbons (e.g., BTX aromatics), the catalysts having improved ethane conversion and/or BTX selectivity, and/or also having decreased selectivity of undesired products (e.g., methane and/or C₉₊ hydrocarbons; etc.). As used herein, “C₉₊ hydrocarbons” refers to hydrocarbons including 9 or more carbon atoms.

Disclosed, in various embodiments, are processes and catalysts for converting ethane to monoaromatic hydrocarbons.

Disclosed herein is a catalyst for converting ethane to monoaromatic hydrocarbons including: a zeolite; cesium oxide, wherein cesium of the cesium oxide is present in an amount of 0.01 to 0.5 weight percent, preferably 0.01 to 0.1 weight percent, more preferably 0.03 to 0.07 weight percent, based on a total weight of the catalyst; platinum oxide, wherein platinum of the platinum oxide is present in an amount of 0.01 to 1 weight percent, preferably 0.01 to 0.5 weight percent, more preferably 0.01 to 0.05 weight percent, based on a total weight of the catalyst; and gallium oxide, wherein gallium of the gallium oxide is present in an amount of 0.01 to 1 weight percent, preferably 0.03 to 0.5 weight percent, more preferably 0.05 to 0.2 weight percent, based on a total weight of the catalyst.

The above described and other features are exemplified by the following figures and detailed description.

Any combination or permutation of embodiments is envisioned. Additional advantageous features, functions and applications of the disclosed processes, methods and catalysts of the present disclosure will be apparent from the description which follows, particularly when read in conjunction with the appended figures. All references listed in this disclosure are hereby incorporated by reference in their entireties.

### BRIEF DESCRIPTION OF THE DRAWINGS

The following figures are exemplary embodiments wherein the like elements are numbered alike.

FIG. 1 is a graph of ethane conversion (percent (%)) versus Time on Stream (TOS) (hours (hr)) showing catalyst performance results of Comparative Example 1.

FIG. 2 is a graph of CH₄ selectivity (%) versus TOS (hr) showing catalyst performance results of Comparative Example 1.

FIG. 3 is a graph of BTX selectivity (%) versus TOS (hr) showing catalyst performance results of Comparative Example 1.

FIG. 4 is a graph of C₉₊ selectivity (%) versus TOS (hr) showing catalyst performance results of Comparative Example 1.

FIG. 5 is a graph of ethane conversion (%) versus TOS (hr) showing catalyst performance results of Example 1.

FIG. 6 is a graph of CH₄ selectivity (%) versus TOS (hr) showing catalyst performance results of Example 1.

FIG. 7 is a graph of BTX selectivity (%) versus TOS (hr) showing catalyst performance results of Example 1.

FIG. 8 is a graph of C₉₊ selectivity (%) versus TOS (hr) showing catalyst performance results of Example 1.

FIG. 9 is a graph of ethane conversion (%), CH₄ selectivity (%), BTX selectivity (%), and C₉₊selectivity (%) showing catalyst performance results of Example 1 and Comparative Example 1.

### DETAILED DESCRIPTION

The exemplary embodiments disclosed herein are illustrative of advantageous catalysts for converting ethane to monoaromatic hydrocarbons (e.g., BTX aromatics), and processes of the present disclosure and methods/techniques thereof. It should be understood, however, that the disclosed embodiments are merely exemplary of the present disclosure, which may be embodied in various forms. Therefore, details disclosed herein with reference to exemplary catalysts for converting ethane to monoaromatic hydrocarbons and associated processes/techniques of fabrication and use are not to be interpreted as limiting, but merely as the basis for teaching one skilled in the art how to make and use the advantageous catalysts for converting ethane to monoaromatic hydrocarbons of the present disclosure.

The present disclosure provides advantageous catalysts for converting ethane to monoaromatic hydrocarbons, and improved processes/methods for utilizing and fabricating the catalysts.

More particularly, the present disclosure provides advantageous catalysts for converting ethane to monoaromatic hydrocarbons (e.g., BTX aromatics), the catalysts having improved ethane conversion and/or BTX selectivity, and/or also having decreased selectivity of undesired products (e.g., methane and/or C9+ hydrocarbons; etc.).

For example, provided is a catalyst with improved performance for converting ethane to BTX. In a non-limiting example, the present disclosure provides that it was surprisingly found that by advantageously modifying a platinum-gallium/zeolite catalyst with cesium can lead to improved performance for converting ethane to BTX (e.g., improved ethane conversion and BTX selectivity and decreased selectivity of an undesired product such as at least one of methane or C₉₊ hydrocarbons) compared to the platinum-gallium/zeolite catalyst without cesium addition. As used herein, “selectivity” refers to a weight percent (wt %), for example, of BTX, methane, or C₉₊ hydrocarbons, present in a total product of the process.

While not wanting to be bound by any theory, it was found that addition of a small amount of cesium to a platinum-gallium/zeolite catalyst can modify strong acid sites of the zeolite and decrease hydrogenolysis during ethane aromatization, which can result in formation of an undesirable product such as methane, and decease alkylation during ethane aromatization, which can result in formation of an undesirable product such as a C₉₊ hydrocarbons. The exemplary cesium-modified platinum-gallium/zeolite catalyst includes small/low amounts of platinum, gallium, and/or cesium to decrease methane formation. An appropriate amount of gallium along with a desirable amount of platinum can decrease formation of methane. The amount of cesium can be controlled to provide a catalyst with relatively high acidity for ethane aromatization, for example, compared to a catalyst for C₃-C₁₂ aromatization having a relatively low acidity. For example, the total acidity of the catalyst measured by Ammonia Temperature Programmed Desorption (NH3-TPD) can be 0.38 to 0.42 millimoles per gram from ammonia, for example, 0.4 millimoles per gram from ammonia.

It is noted that the acidity of a catalyst for converting ethane to BTX can be greater than the acidity of a light naphtha aromatization (LNA) catalyst. Typically, an LNA catalyst includes a greater amount of cesium as compared to an ethane aromatization catalyst, and an LNA catalyst also includes germanium.

The present disclosure provides that an exemplary catalyst for converting ethane to monoaromatic hydrocarbons (e.g., BTX aromatics) can include a zeolite; cesium oxide, wherein cesium of the cesium oxide is present in an amount of 0.01 to 0.5 wt %, preferably 0.01 to 0.1 wt %, more preferably 0.03 to 0.07 wt %, based on a total weight of the catalyst; platinum oxide, wherein platinum of the platinum oxide is present in an amount of 0.01 to 1 wt %, preferably 0.01 to 0.5 wt %, more preferably 0.01 to 0.05 wt %, based on a total weight of the catalyst; and gallium oxide, wherein gallium of the gallium oxide is present in an amount of 0.01 to 1 wt %, preferably 0.03 to 0.5 wt %, more preferably 0.05 to 0.2 wt %, based on a total weight of the catalyst.

For example, an exemplary zeolite of the cesium-modified platinum-gallium/zeolite catalyst can be an aluminosilicate zeolite. The zeolite can be, for example and without limitation, ZSM-5, ZSM-8, ZSM-11, ZSM-12, or ZSM-35. The zeolite can be ZSM-5. The zeolite can be H-ZSM-5. The zeolite can have a SiO₂/Al₂O₃ mole ratio of 1 to 60, preferably 10 to 50, more preferably 20 to 40. It is noted that the zeolite can have a SiO₂/Al₂O₃ mole ratio of 30.

The catalyst can include from 10 to 99.9 wt % of zeolite, preferably from 30 to 99.9 wt %, based on a total weight of the catalyst. The zeolite can be converted to H⁺ form (e.g., H-ZSM-5) to provide acidity to help catalyze the dehydroaromatization reaction, which can be accomplished by calcining the ammonium form of the zeolite, for example, at a temperature of at least 400° C., for example, 500 to 700° C., for a period of time of, for example, 5 to 15 hours.

The catalyst can include a binder such as alumina hydrate. The binder can include an aluminum- or silicon-containing material such as silica, alumina, clay, aluminum phosphate, silica-alumina, or combinations including at least one of the foregoing. The binder can include oxides of magnesium, titanium, zirconium, thorium, silicon, boron, and mixtures thereof; a clay, e.g., kaolin or montmorillonite; carbon, e.g., carbon black, graphite, activated carbon, polymers or charcoal; a metal carbide or nitride, e.g., molybdenum carbide, silicon carbide or tungsten nitride; a metal oxide hydroxide, e.g., boehmite; or a combination comprising at least one of the foregoing. For example, the amount of zeolite can be 50 to 95 wt %, 60 to 95 wt %, or 70 to 90 wt %, based on total weight of the zeolite and the binder, and the amount of binder can be 5 to 50 wt %, 5 to 40 wt %, or 10 to 30 wt %, based on total weight of the zeolite and the binder.

An exemplary method of forming the catalyst can include mixing a zeolite, binder, and peptizing agent to form a mixture. The peptizing agent can be an acid such as an organic acid or an inorganic acid. Exemplary organic acids include formic acid (HCO₂H), acetic acid (CH₃COOH), propionic acid, butyric acid, oxalic acid, lactic acid, malic acid, citric acid, benzoic acid, carbonic acid, uric acid, or a combination comprising at least one of the foregoing, preferably acetic acid (CH₃COOH). Exemplary inorganic acids include nitric acid (HNO₃), phosphoric acid (H₃PO₄), or a combination comprising at least one of the foregoing. The mixture can then be extruded to form an extrudate.

Hydroxypropyl methylcellulose, hydroxyethyl methyl cellulose, hydroxyethyl cellulose, or a combination comprising at least one of the foregoing can be added during extrudate preparation to increase porosity. For example, the amount of hydroxypropyl methylcellulose, hydroxyethyl methyl cellulose, hydroxyethyl cellulose, or a combination comprising at least one of the foregoing can be 1 to 5 wt %, based on total weight of the extrudate.

It is noted that the exemplary catalyst can be an extrudate, although the present disclosure is not limited thereto. Rather, it is noted that the catalyst can take a variety of shapes and/or forms. The final shaped catalyst could be in the form of cylindrical pellets, rings or spheres. Extrudate size can be based on reactor dimensions (e.g., of a fixed bed reactor) to maintain a uniform plug-flow condition. Following extrusion, the catalyst can be calcined, e.g., a temperature greater than 400° C., for example, 500° C. to 700° C. for a period of time, for example, 5 to 15 hours.

Metal loading can be done, for example, by incipient wetness impregnation. The desired amount of metal precursor (e.g., platinum-containing precursor or gallium-containing precursor) can be dissolved in water and impregnated onto the zeolite/binder structure. The amount of water can be based upon pore volume of the zeolite/binder calcined structure.

For example, platinum and gallium can be incorporated onto a zeolite by incipient wetness impregnation to provide a platinum-gallium/zeolite; and cesium can be incorporated onto the platinum-gallium/zeolite by incipient wetness impregnation to provide the catalyst. The platinum and gallium can be incorporated onto the zeolite by incipient wetness impregnation in a single step. The platinum and gallium can be present on the zeolite in close proximity. The close proximity of platinum and gallium allows platinum to initiate dehydrogenation of ethane and gallium to facilitate further dehydrogenation to provide an effective catalyst for ethane aromatization, which can be difficult to activate, for example, compared to C₃-C₁₂ aromatization. Each of the platinum, gallium, and cesium components can be present on the surface of the zeolite in the form of a metal oxide, e.g., platinum oxide, gallium oxide, and cesium oxide, respectively. By contrast, metal incorporated by, for example, hydrothermal synthesis or ion exchange, would be present in cationic form.

The present disclosure provides that a process for converting ethane to monoaromatic hydrocarbons (BTX) can include providing the catalyst and contacting ethane with the catalyst. Process conditions for converting ethane to monoaromatic hydrocarbons (BTX) can include contacting the ethane with the catalyst at a pressure of 1 to 900 kilopascals (kPa), a temperature of 100 to 900° C., and a gas hourly space velocity (GHSV) (of ethane) of 1,000 to 9,000 milliliters per gram of catalyst per hour (ml·(g of Cat)⁻¹·h⁻¹); preferably a pressure of 100 to 800 kPa, a temperature of 200 to 800° C., and a GHSV of 2,000 to 8,000 ml·(g of Cat)⁻¹·h⁻¹; more preferably a pressure of 200 to 700 kPa, a temperature of 300 to 700 ° C., and a GHSV of 3,000 to 7,000 ml·(g of Cat)⁻¹·h⁻¹.

The exemplary catalyst (e.g., cesium-modified platinum-gallium/zeolite catalyst) can exhibit improved ethane conversion and/or BTX selectivity and/or decreased selectivity of an undesired product (at least one of methane or C₉₊ hydrocarbons) compared to a platinum-gallium/zeolite catalyst without cesium addition. For example, greater than 42 wt %, preferably greater than 42.5 wt %, more preferably greater than 43 wt %, of the ethane can be converted. Conversion of ethane can be calculated as follows:

((weight of ethane fed to the reactor)−(weight of ethane exiting the reactor))/(weight of ethane fed to the reactor)×100 wt %.

A product of the process can include less than 25 wt %, preferably less than 24 wt %, more preferably less than 23 wt %, methane at an ethane conversion of greater than 42 weight percent, preferably greater than 42.5 weight percent, more preferably greater than 43 weight percent. A product of the process can include greater than 53 wt %, preferably greater than 53.3 wt %, more preferably greater than 53.6 wt %, benzene, toluene, and xylenes at an ethane conversion of greater than 42 weight percent, preferably greater than 42.5 weight percent, more preferably greater than 43 weight percent. A product of the process can include less than 10 wt %, preferably less than 9.7 wt %, more preferably less than 9.4 wt %, C₉₊ hydrocarbons at an ethane conversion of greater than 42 weight percent, preferably greater than 42.5 weight percent, more preferably greater than 43 weight percent.

At an ethane conversion of greater than 42 weight percent, preferably greater than 42.5 weight percent, more preferably greater than 43 weight percent, a product of the process can include at least two of less than 25 wt %, preferably less than 24 wt %, more preferably less than 23 wt %, methane; greater than 53 wt %, preferably greater than 53.3 wt %, more preferably greater than 53.6 wt %, benzene, toluene, and xylenes; or less than 10 wt %, preferably less than 9.7 wt %, more preferably less than 9.4 wt %, C₉₊ hydrocarbons. For example, at an ethane conversion of greater than 42 weight percent, preferably greater than 42.5 weight percent, more preferably greater than 43 weight percent, a product of the process can include less than 25 wt %, preferably less than 24 wt %, more preferably less than 23 wt %, methane; greater than 53 wt %, preferably greater than 53.3 wt %, more preferably greater than 53.6 wt %, benzene, toluene, and xylenes; and less than 10 wt %, preferably less than 9.7 wt %, more preferably less than 9.4 wt %, C₉₊ hydrocarbons.

The exemplary cesium-modified platinum-gallium/zeolite catalyst can exhibit a lower deactivation rate, Kd, compared to a platinum gallium/zeolite catalyst. For example, a deactivation rate of the cesium-modified platinum-gallium/zeolite catalyst can be less than 2.4 percent per hour, preferably less than 2.35 percent per hour, more preferably less than 2.3 percent per hour, calculated by dividing a difference in ethane conversion at the first hour and the fifth hour by 4 hours.

This disclosure is further illustrated by the following examples, which are non-limiting.

### EXAMPLES

The following components listed in Table 1 were used in the examples. Unless specifically indicated otherwise, the amount of each component is in weight percent (wt %) in the following examples, based on the total weight of the composition.

TABLE 1

| Category | Characteristics | Tradename | Manufacturer |
|---|---|---|---|
| Zeolite | NH₄-ZSM-5, 30:1 SiO₂:Al₂O₃ mole | CBV 3024E, | Zeolyst |
| | ratio, surface area: 405 square | | |
| | meters per gram (m²/g), Na₂O | | |
| | content: 0.05 wt %, particle size: | | |
| | about 200 to 350 nanometers (nm) | | |
| Alumina | AlOOH, surface area: 250 m²/g, | Pural SCF-55 | Sasol |
| hydrate | Na₂O content: 0.002 wt %, Pore | | |
| | volume: 0.5 milliliters per gram | | |
| | (mL/g) | | |
| Hydroxypropyl | C₅₆H₁₀₈O₃₀ | | SD Fine |
| methylcellulose | | | chemicals |
| Peptizing agent | CH₃COOH (American Chemical | | Sigma |
| | Society reagent, ≥99.7%) | | Aldrich |

### Support Preparation

Step 1: H-ZSM-5 Preparation

About 200 grams (g) of NH₃ form of zeolite was calcined at a temperature of 600° C. for 10 hours in a tubular quartz reactor to provide H-ZSM-5.

Step 2: Extrudate Preparation

80 g of H-ZSM-5 was mixed with 20 g of alumina hydrate and 6.2 g of hydroxypropyl methylcellulose. 3 wt % peptizing agent was added to the mixture in step wise manner. 1.5 millimeter extrudates were made using the mixture with a laboratory single screw extruder. The extrudates were dried in air for 2 hours followed by oven drying at 120° C. for 6 hours and calcined at 600° C. for 10 hours in a tubular quartz reactor

Catalyst Preparation

### Comparative Example 1

A solution having 500 parts per million by weight (ppmw) platinum was prepared using chloroplatinic acid and a solution having 1,500 ppmw gallium was prepared using gallium nitrate. Amounts of platinum and gallium salts dissolved in the water was based on pore volume calculation, with pore volume of the dried extrudate measured by deionized water. Using the solutions including platinum and gallium, platinum and gallium were impregnated onto 25 g of calcined extrudates by incipient wetness method, which included drying at 300° C. for 2 hours followed by cooling to room temperature under the flow of dry air. The impregnated catalyst was dried in air for 2 hours followed by oven drying at 120° C. for 6 hours and calcined at 550° C. for 6 hours to form a platinum-gallium/ZSM-5 catalyst. Inductively coupled plasma analysis indicated that the catalyst of Comparative Example 1 included 240 ppmw platinum and 1,500 ppmw gallium.

### Comparative Example 2

A solution having 500 ppmw platinum was prepared using chloroplatinic acid and a solution having 1,500 ppmw gallium was prepared using gallium nitrate. Amounts of platinum and gallium salts dissolved in the water was based on pore volume calculation, with pore volume of the dried extrudate measured by deionized water. Using the solutions including platinum and gallium, platinum and gallium were impregnated onto 25 g of calcined extrudates by incipient wetness method, which included drying at 300° C. for 2 hours followed by cooling to room temperature under the flow of dry air. The impregnated catalyst was dried in air for 2 hours followed by oven drying at 120° C. for 6 hours and calcined at 550° C. for 6 hours to form a platinum-gallium/ZSM-5 catalyst. Inductively coupled plasma analysis indicated that the catalyst of Comparative Example 2 included 240 ppmw platinum and 5,000 ppmw gallium.

### Performance Evaluation of Comparative Example 1 and Comparative Example 2

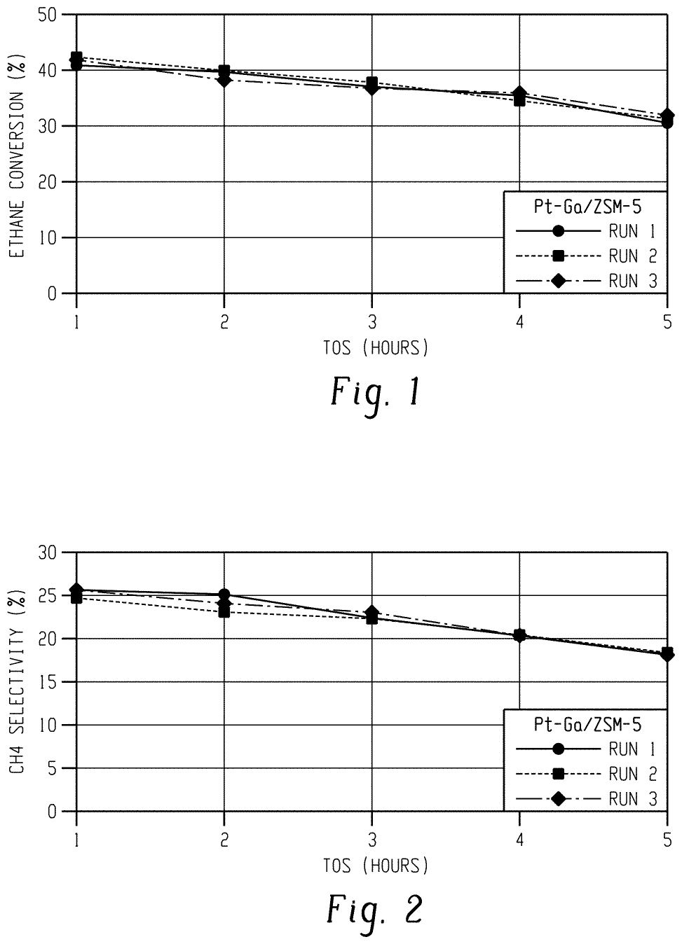

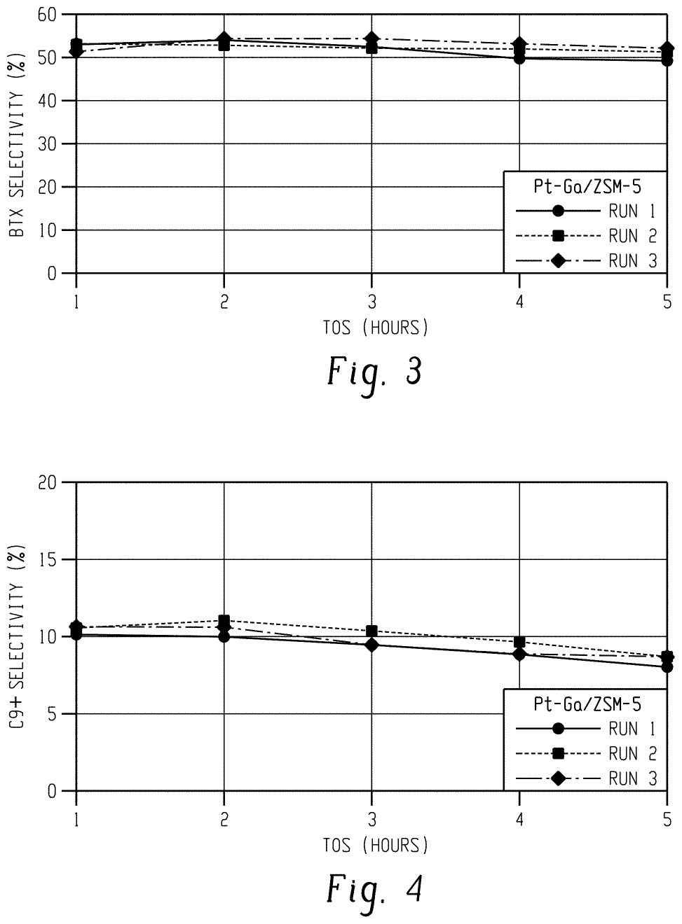

Performance evaluation of 6 g of each of the platinum-gallium/ZSM-5 catalysts of Comparative Example 1 and Comparative Example 2 was done in a fixed bed reactor at 630° C., pressure of 5 atmospheres (atm) (506.625 kilopascals (kPa)), and ethane gas hourly space velocity (GHSV) of 5,000 ml·g⁻¹ cat·h⁻¹. The products were analyzed using gas chromatography. FIGS. 1-4 are graphs of ethane conversion (percent (%)) versus Time on Stream (TOS) (hours (hr)), CH₄ selectivity (%) versus TOS (hr), BTX selectivity (%) versus TOS (hr), and C₉₊ selectivity (%) versus TOS (hr), respectively, showing catalyst performance results of Comparative Example 1. The catalyst performance results of Comparative Example 1 and Comparative Example 2 at 1 hour, at which maximum conversion was achieved, are provided in Table 2.

TABLE 2

| | | | | C₉₊ |
|---|---|---|---|---|
| | Ethane | CH₄ | BTX | hydrocarbon |
| | Conversion | selectivity | selectivity | selectivity |
| Comparative | 42 wt % | 25 wt % | 53 wt % | 10 wt % |
| Example 1 | | | | |
| Comparative | 38 wt % | 30 wt % | 50 wt % | 14 wt % |
| Example 2 | | | | |

Comparative Example 1 exhibited favorable results in terms of ethane conversion, BTX selectivity, methane selectivity, and C₉₊ hydrocarbon selectively over Comparative Example 2. Accordingly, the catalyst of Comparative Example 1 was used as to prepare a catalyst of Example 1.

### Example 1

A cesium solution having 500 ppmw cesium was prepared using cesium nitrate. Using the solution including cesium, cesium was impregnated on the platinum-gallium/ZSM-5 catalyst of Comparative Example 1 by incipient wetness method, which included drying at 300° C. for 2 hours followed by cooling to room temperature under the flow of dry air. The impregnated catalyst was dried in air for 2 hours followed by oven drying at 120° C. for 6 hours and calcined at 550° C. for 6 hours. Inductively coupled plasma analysis indicated that the catalyst of Example 1 included 240 ppmw platinum, 1,500 ppmw gallium, and 500 ppmw cesium.

### Performance Evaluation of Example 1

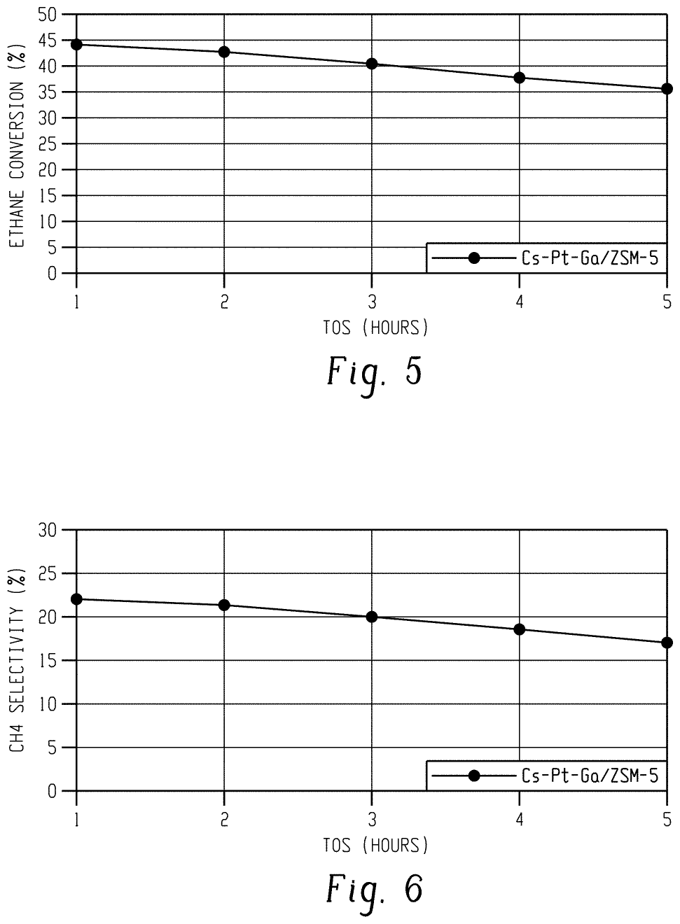

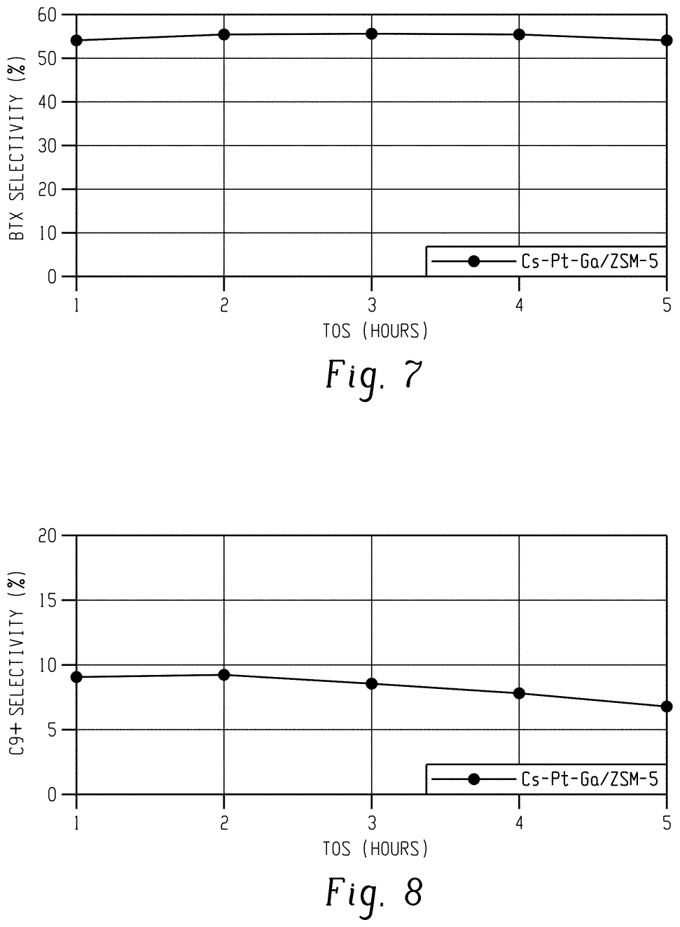

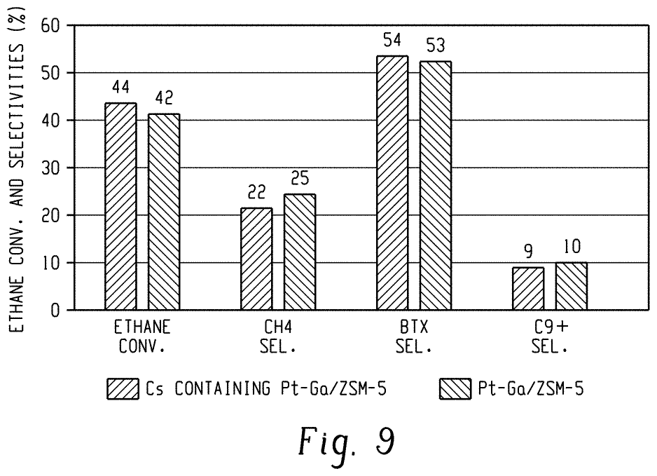

Performance evaluation of 6 g of the cesium-modified platinum-gallium/ZSM-5 catalyst of Example 1 was done in a fixed bed reactor at 630° C., pressure of 5 atm (506.625 kPa), and ethane GHSV of 5,000 ml·g⁻¹ cat·h⁻¹. The products were analyzed using gas chromatography. FIGS. 5-8 are graphs of ethane conversion (%) versus TOS (hr), CH₄ selectivity (%) versus TOS (hr), BTX selectivity (%) versus TOS (hr), and C₉₊ selectivity (%) versus TOS (hr), respectively, showing catalyst performance results of Example 1, and . The catalyst performance results of Example 1 at 1 hour, at which maximum conversion was achieved, are provided in Table 3 and FIG. 9, which also includes catalyst performance results of Comparative Example 1 at 1 hour.

TABLE 3

| | | | | | C₉₊ |
|---|---|---|---|---|---|
| | | Ethane | CH₄ | BTX | hydrocarbon |
| | | Conversion | selectivity | selectivity | selectivity |
| Comparative | Platinum- | 42 wt % | 25 wt % | 53 wt % | 10 wt % |
| Example 1 | gallium/ZSM-5 catalyst | | | | |
| Example 1 | Cesium-modified | 44 wt % | 22 wt % | 54 wt % | 9 wt % |
| | platinum- | | | | |
| | gallium/ZSM-5 catalyst | | | | |

The cesium-modified platinum-gallium/ZSM-5 catalyst exhibited improved ethane conversion and BTX (benzene, toluene, and xylenes) selectivity and decreased selectivity of an undesired product such as at least one of methane or C₉₊ hydrocarbons (e.g., trimethyl benzene, naphthalene, and substituted naphthalene) in comparison with platinum-gallium/ZSM-5 catalyst.

The cesium-modified platinum-gallium/ZSM-5 catalyst also exhibited a lower deactivation rate, K_{d}, compared to the platinum-gallium/ZSM-5 catalyst. The K_{d} for the platinum-gallium/ZSM-5 catalyst was 2.4 percent per hour and for the cesium-modified platinum-gallium/ZSM-5 catalyst the K_{d} was 2.2 percent per hour. K_{d} was calculated by dividing a difference in ethane conversion at the first hour and the fifth hour by the time difference (i.e., 4 hours).

This disclosure further encompasses the following aspects.

Aspect 1. A catalyst for converting ethane to monoaromatic hydrocarbons comprising: a zeolite; cesium oxide, wherein cesium of the cesium oxide is present in an amount of 0.01 to 0.5 weight percent, preferably 0.01 to 0.1 weight percent, more preferably 0.03 to 0.07 weight percent, based on a total weight of the catalyst; platinum oxide, wherein platinum of the platinum oxide is present in an amount of 0.01 to 1 weight percent, preferably 0.01 to 0.5 weight percent, more preferably 0.01 to 0.05 weight percent, based on a total weight of the catalyst; and gallium oxide, wherein gallium of the gallium oxide is present in an amount of 0.01 to 1 weight percent, preferably 0.03 to 0.5 weight percent, more preferably 0.05 to 0.2 weight percent, based on a total weight of the catalyst.

Aspect 2. The catalyst of Aspect 1, wherein the zeolite comprises an aluminosilicate zeolite.

Aspect 3. The catalyst of Aspect 2, wherein the zeolite comprises ZSM-5.

Aspect 4. The catalyst of Aspect 3, wherein the zeolite comprises H-ZSM-5.

Aspect 5. The catalyst of any of Aspects 2-4, wherein the zeolite has a SiO₂/Al₂O₃ mole ratio of 1 to 60, preferably 10 to 50, more preferably 20 to 40.

Aspect 6. The catalyst of any of Aspects 1-5, wherein the zeolite is an extrudate.

Aspect 7. The catalyst of any of Aspects 1-6, further comprising a binder in an amount of 5 to 50 weight percent, preferably 5 to 40 weight percent, more preferably 10 to 30 weight percent, based on a total weight of the zeolite and the binder.

Aspect 8. A process for converting ethane to monoaromatic hydrocarbons, the process comprising: providing the catalyst of any of Aspects 1-7; and contacting ethane with the catalyst, wherein the monoaromatic hydrocarbons comprise benzene, toluene, xylene, or a combination comprising at least one of the foregoing.

Aspect 9. The process of Aspect 8, wherein the ethane is contacted with the catalyst at a pressure of 1 to 900 kilopascals, a temperature of 100 to 900° C., and a gas hourly space velocity of 1,000 to 9,000 milliliters per gram of catalyst per hour; preferably a pressure of 100 to 800 kilopascals, a temperature of 200 to 800° C., and a gas hourly space velocity of 2,000 to 8,000 milliliters per gram of catalyst per hour; more preferably a pressure of 200 to 700 kilopascals, a temperature of 300 to 700° C., and a gas hourly space velocity of 3,000 to 7,000 milliliters per gram of catalyst per hour.

Aspect 10. The process of Aspect 9, wherein greater than 42 weight percent, preferably greater than 42.5 weight percent, more preferably greater than 43 weight percent, of the ethane is converted.

Aspect 11. The process of Aspect 10, wherein a product of the process comprises less than 25 weight percent, preferably less than 24 weight percent, more preferably less than 23 weight percent, methane at an ethane conversion of greater than 42 weight percent, preferably greater than 42.5 weight percent, more preferably greater than 43 weight percent.

Aspect 12. The process of Aspect 10 or Aspect 11, wherein a product of the process comprises greater than 53 weight percent, preferably greater than 53.3 weight percent, more preferably greater than 53.6 weight percent, benzene, toluene, and xylenes at an ethane conversion of greater than 42 weight percent, preferably greater than 42.5 weight percent, more preferably greater than 43 weight percent.

Aspect 13. The process of any of Aspects 10-12, wherein a product of the process comprises less than 10 weight percent, preferably less than 9.7 weight percent, more preferably less than 9.4 weight percent, C₉₊ hydrocarbons at an ethane conversion of greater than 42 weight percent, preferably greater than 42.5 weight percent, more preferably greater than 43 weight percent.

Aspect 14. The process of Aspect 10, wherein, at an ethane conversion of greater than 42 weight percent, preferably greater than 42.5 weight percent, more preferably greater than 43 weight percent, a product of the process comprises at least two of: less than 25 weight percent, preferably less than 24 weight percent, more preferably less than 23 weight percent, methane; greater than 53 weight percent, preferably greater than 53.3 weight percent, more preferably greater than 53.6 weight percent, benzene, toluene, and xylenes; or less than 10 weight percent, preferably less than 9.7 weight percent, more preferably less than 9.4 weight percent, C₉₊ hydrocarbons.

Aspect 15. The process of any of Aspects 9-14, wherein a deactivation rate of the catalyst is less than 2.4 percent per hour, preferably less than 2.35 percent per hour, more preferably less than 2.3 percent per hour, calculated by dividing a difference in ethane conversion at the first hour and the fifth hour by 4 hours.

Aspect 16. A method for preparing the catalyst of any of Aspects 1-7, the method comprising: incorporating platinum and gallium onto a zeolite by incipient wetness impregnation to provide a platinum-gallium/zeolite; and incorporating cesium onto the platinum-gallium/zeolite by incipient wetness impregnation to provide the catalyst, wherein the cesium is present in an amount of 0.01 to 0.5 weight percent, preferably 0.01 to 0.1 weight percent, more preferably 0.03 to 0.07 weight percent, based on a total weight of the catalyst, wherein the platinum is present in an amount of 0.01 to 1 weight percent, preferably 0.01 to 0.5 weight percent, more preferably 0.01 to 0.05 weight percent, based on a total weight of the catalyst, and wherein the gallium is present in an amount of 0.01 to 1 weight percent, preferably 0.03 to 0.5 weight percent, more preferably 0.05 to 0.2 weight percent, based on a total weight of the catalyst.

Aspect 17. The method of Aspect 16, wherein: incorporating platinum and gallium onto the zeolite comprises incorporating platinum oxide and gallium oxide onto the zeolite; and incorporating cesium onto the platinum-gallium/zeolite comprises incorporating cesium oxide onto the platinum-gallium/zeolite.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other (e.g., ranges of “up to 25 wt %, or, more specifically, 5 wt % to 20 wt %”, is inclusive of the endpoints and all intermediate values of the ranges of “5 wt % to 25 wt %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

Although the processes, methods and catalysts of the present disclosure have been described with reference to exemplary embodiments thereof, the present disclosure is not limited to such exemplary embodiments and/or implementations. Rather, the processes, methods and catalysts of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

1. A catalyst for converting ethane to monoaromatic hydrocarbons comprising:
a zeolite;
cesium oxide, wherein cesium of the cesium oxide is present in an amount of 0.01 to 0.5 weight percent, based on a total weight of the catalyst;
platinum oxide, wherein platinum of the platinum oxide is present in an amount of 0.01 to 1 weight percent, based on a total weight of the catalyst; and
gallium oxide, wherein gallium of the gallium oxide is present in an amount of 0.01 to 1 weight percent, based on a total weight of the catalyst.

2. The catalyst of claim 1, wherein the zeolite comprises an aluminosilicate zeolite.

3. The catalyst of claim 2, wherein the zeolite comprises ZSM-5.

4. The catalyst of claim 3, wherein the zeolite comprises H-ZSM-5.

5. The catalyst of claim 2, wherein the zeolite has a SiO₂/Al₂O₃ mole ratio of 1 to 60.

6. The catalyst of claim 1, wherein the zeolite is an extrudate.

7. The catalyst of claim 1, further comprising a binder in an amount of 5 to 50 weight percent, based on a total weight of the zeolite and the binder.

8. A process for converting ethane to monoaromatic hydrocarbons, the process comprising:
providing the catalyst of claim 1; and
contacting ethane with the catalyst,
wherein the monoaromatic hydrocarbons comprise benzene, toluene, xylene, or a combination comprising at least one of the foregoing.

9. The process of claim 8, wherein the ethane is contacted with the catalyst at a pressure of 1 to 900 kilopascals, a temperature of 100 to 900° C., and a gas hourly space velocity of 1,000 to 9,000 milliliters per gram of catalyst per hour.

10. The process of claim 9, wherein greater than 42 weight percent of the ethane is converted.

11. The process of claim 10, wherein a product of the process comprises less than 25 weight percent methane at an ethane conversion of greater than 42 weight percent.

12. The process of claim 10, wherein a product of the process comprises greater than 53 weight percent benzene, toluene, and xylenes at an ethane conversion of greater than 42 weight percent.

13. The process of claim 10, wherein a product of the process comprises less than 10 weight percent C₉₊ hydrocarbons at an ethane conversion of greater than 42 weight percent.

14. The process of claim 10, wherein, at an ethane conversion of greater than 42 weight percent, a product of the process comprises at least two of:
less than 25 weight percent methane;
greater than 53 weight percent benzene, toluene, and xylenes; or
less than 10 weight percent C₉₊ hydrocarbons.

15. The process of claim 9, wherein a deactivation rate of the catalyst is less than 2.4 percent per hour, calculated by dividing a difference in ethane conversion at the first hour and the fifth hour by 4 hours.

16. A method for preparing the catalyst of claim 1, the method comprising:
incorporating platinum and gallium onto a zeolite by incipient wetness impregnation to provide a platinum-gallium/zeolite; and
incorporating cesium onto the platinum-gallium/zeolite by incipient wetness impregnation to provide the catalyst,
wherein the cesium is present in an amount of 0.01 to 0.5 weight percent, based on a total weight of the catalyst,
wherein the platinum is present in an amount of 0.01 to 1 weight percent, based on a total weight of the catalyst, and
wherein the gallium is present in an amount of 0.01 to 1 weight percent, based on a total weight of the catalyst.

17. The method of claim 16, wherein:
incorporating platinum and gallium onto the zeolite comprises incorporating platinum oxide and gallium oxide onto the zeolite; and
incorporating cesium onto the platinum-gallium/zeolite comprises incorporating cesium oxide onto the platinum-gallium/zeolite.
